(19) United States
(12) Patent Application Publication
(10) Pub. No.: US 2026/0207444 A1
(43) Pub. Date: Jul. 23, 2026

(54) SOLID POWDER COSMETIC

(71) Applicant: KOSE Corporation, Tpkyo (JP)

(72) Inventors: Daigo Arita, Tokyo (JP); Wataru Maruki, Tokyo (JP)

(73) Assignee: KOSE Corporation, Tpkyo (JP)

(21) Appl. No.: 19/140,291

(22) PCT Filed: Dec. 27, 2023
(86) PCT No.: PCT/JP2023/046858

§ 371 Date: Jun. 17, 2025

(30) Foreign Application Priority Data
Dec. 28, 2022 (JP) 2022-212766

(51) Int. Cl. A61K 8/02; A61K 8/19; A61K 8/25; A61Q 1/02

(52) U.S. Cl. CPC A61K 8/0266; A61K 8/19; A61K 8/25; A61Q 1/02; A61K 2800/43; A61K 2800/621

(57) ABSTRACT

To provide a solid powder cosmetic excellent in all of a matte texture, absence of an appearance color difference before and after use, filling moldability, and easiness of scooping, and a method for producing the same.

A solid powder cosmetic being obtained by adding a solvent to a cosmetic base containing the following components (A) and (B);
(A) a non-brilliant powder in which (a-2) a plate-like powder is coated with (a-1) a chromatic iron oxide, and
(B) a non-volatile oil
to form a slurry, filling a container with the slurry, and then removing the solvent,
in which a chromatic iron oxide not coating a powder component is not contained or a content of the iron oxide is 15 mass % or less with respect to a total amount of the cosmetic,
a brilliant powder is not contained or a content of the brilliant powder is 15 mass % or less with respect to the total amount of the cosmetic, and
a mass content ratio of the (a-1) and the chromatic iron oxide not coating the powder component, i.e., (a-1):(the chromatic iron oxide not coating the powder component) is 100:0 to 20:80.

### TECHNICAL FIELD

The present invention relates to a solid powder cosmetic and a method for producing a solid powder cosmetic.

### BACKGROUND ART

The solid powder cosmetic is composed of a powder component and an oily component, and is used in makeup cosmetics such as eye shadow, blusher, and foundation. Generally, as a method for molding a solid powder cosmetic, a dry method in which a powder component and an oily component are mixed and the mixture is filled in a mold and pressed to mold a solid powder cosmetic, and a wet method in which a solvent is mixed with a powder component and an oily component to form a slurry, the obtained slurry is filled in a mold and pressed, and then the solvent is dried and removed to mold a solid powder cosmetic are known. While a soft and moist feel of use is obtained in a solid powder cosmetic using a wet method, a difference in appearance color before and after use may occur due to a difference in orientation of the powder between the pressed surface and the inside of the cosmetic.

In order to solve such a problem, for example, there have been proposed a solid powder cosmetic blended with silica having an oil absorption of 100 ml/100 g or more and a colored pigment, the solid powder cosmetic being produced by adding a volatile solvent to the powder cosmetic to form a slurry, then filling a container with the slurry, and then removing the solvent (Patent Literature 1), a solid powder cosmetic in which contents of a powder component and an oily component are 70 to 95 mass % and 5 to 30 mass %, respectively, based on a total amount of the solid powder cosmetic, the oily component containing a hardened castor oil fatty acid ester and heavy liquid isoparaffin (Patent Literature 2), and the like.

### CITATION LIST

### Patent Literatures

Patent Literature 1: JP 2011-105626 A
Patent Literature 2: JP 2019-119716 A

### SUMMARY OF INVENTION

However, when the above technique is used, a solid powder cosmetic having a matte appearance may be poor in filling moldability and easiness of scooping (when a user takes a cosmetic with a finger, an applicator, or the like, the cosmetic attached to the finger, the applicator, or the like is suitable).

Therefore, an object of the present invention is to provide a solid powder cosmetic which has no or little appearance color difference before and after use and is excellent in all of a matte texture, filling moldability, and easiness of scooping, and a method for producing the same.

As a result of intensive studies, the present inventors have found that, in a solid powder cosmetic using a wet method, by combining a non-brilliant powder in which a plate-like powder is coated with a chromatic iron oxide and a non-volatile oil agent, a solid powder cosmetic excellent in all of a matte texture, absence of an appearance color difference before and after use, filling moldability, and easiness of scooping is obtained, thereby completing the present invention.

That is, the present invention provides the following.

[1]

A solid powder cosmetic being obtained by adding a solvent to a cosmetic base containing the following components (A) and (B);
(A) a non-brilliant powder in which (a-2) a plate-like powder is coated with (a-1) a chromatic iron oxide, and
(B) a non-volatile oil
to form a slurry, filling a container with the slurry, and then removing the solvent,
in which a chromatic iron oxide not coating a powder component is not contained or a content of the iron oxide is 15 mass % or less with respect to a total amount of the cosmetic,
a brilliant powder is not contained or a content of the brilliant powder is 15 mass % or less with respect to the total amount of the cosmetic, and
a mass content ratio of the (a-1) and the chromatic iron oxide not coating the powder component, i.e., (a-1):(the chromatic iron oxide not coating the powder component) is 100:0 to 20:80.
   [2]

The solid powder cosmetic according to [1], wherein the (a-2) is one or two or more kinds selected from mica and synthetic phlogopite.

[3]

The solid powder cosmetic according to [1] or [2], wherein the brilliant powder is not substantially contained or the content of the brilliant powder with respect to the total amount of the cosmetic is 5% or less.

[4]

The solid powder cosmetic according to [1] or [2], wherein the component (B) is one or two or more kinds selected from the group consisting of a hydrocarbon oil, a silicone oil, and an ester oil.

[5]

A method for producing a solid powder cosmetic obtained by adding a solvent to a cosmetic base containing the following components (A) and (B);
(A) a non-brilliant powder in which (a-2) a plate-like powder is coated with (a-1) a chromatic iron oxide, and
(B) a non-volatile oil
to form a slurry, filling a container with the slurry, and then removing the solvent,
in which a chromatic iron oxide not coating a powder component is not contained or a content of the iron oxide is 15 mass % or less with respect to a total amount of the cosmetic,
a brilliant powder is not contained or a content of the brilliant powder is 15 mass % or less with respect to the total amount of the cosmetic, and
a mass content ratio of the (a-1) and the chromatic iron oxide not coating the powder component, i.e., (a-1):(the chromatic iron oxide not coating the powder component) is 100:0 to 20:80.

An aspect of the present invention is
a solid powder cosmetic being obtained by adding a solvent to a cosmetic base containing the following components (A) and (B);
(A) a non-brilliant powder in which (a-2) a plate-like powder is coated with (a-1) red iron oxide and/or yellow iron oxide, and
(B) a non-volatile oil
to form a slurry, filling a container with the slurry, and then removing the solvent,
in which red iron oxide and/or yellow iron oxide not coating a powder component is not contained or a content of the iron oxide is 15 mass % or less with respect to a total amount of the cosmetic,
a brilliant powder is not contained or a content of the brilliant powder is 15 mass % or less with respect to the total amount of the cosmetic, and a mass content ratio of the (a-1) and the red iron oxide and/or yellow iron oxide not coating the powder component, i.e., (a-1):(the red iron oxide and/or yellow iron oxide not coating the powder component) is 100:0 to 20:80.

Another aspect of the present invention is
a solid powder cosmetic being obtained by adding a solvent to a cosmetic base containing the following components (A) and (B);
(A) a non-brilliant powder in which (a-2) synthetic phlogopite is coated with (a-1) red iron oxide and/or yellow iron oxide, and
(B) a non-volatile oil
to form a slurry, filling a container with the slurry, and then removing the solvent,
in which red iron oxide and/or yellow iron oxide (provided that the component (A) is excluded) is not contained or a content of the iron oxide (red iron oxide and/or yellow iron oxide) is 15 mass % or less with respect to a total amount of the cosmetic,
a brilliant powder is not contained or a content of the brilliant powder is 15 mass % or less with respect to the total amount of the cosmetic, and a mass content ratio of the (a-1) and the red iron oxide and/or yellow iron oxide not coating the powder component, i.e., (a-1):(the red iron oxide and/or yellow iron oxide not coating the powder component) is 100:0 to 20:80.

### DESCRIPTION OF EMBODIMENTS

Hereinafter, embodiments of the present invention will be described. The present invention is not limited only to the following embodiments. In the present specification, the phrase “X to Y” indicating a range includes X and Y and means “X or more and Y or less”. In the present specification, the percentage (%) is expressed by mass unless otherwise specified. In the present specification, in the case of describing a content of a certain component, when there are two or more kinds of the components, the content refers to the total amount thereof.

A solid powder cosmetic of the present invention is excellent in a matte texture, absence of an appearance color difference before and after use (hereinafter, also simply referred to as absence of a color difference in appearance), filling moldability, and easiness of scooping.

In the case of providing a cosmetic having saturation, a chromatic brilliant powder can be used, but since the cosmetic has brilliance, the blending amount of the chromatic brilliant powder is limited in order to impart a matte texture to the cosmetic. In the case of similarly providing a cosmetic having saturation, an iron oxide having saturation can be used, but when such an iron oxide is used as it is in a wet-molded solid powder cosmetic, a color difference in appearance before and after use significantly occurs, and moldability is not sufficient (Comparative Example 2 described later). The present inventors presume that this is because the orientation of the powder on the pressed surface and the inside of the cosmetic is different due to the blending of the iron oxide derived from the production method of the wet method. The present inventors have found such a problem, and as a means for solving the problem, have found that the above effect is exhibited by using a non-brilliant powder obtained by coating a plate-like powder with a chromatic iron oxide as a main component in a chromatic iron oxide in a solid powder cosmetic of a wet method (a method of adding a solvent to a cosmetic base to form a slurry, filling the slurry in a container, and then removing the solvent to obtain a solid powder cosmetic), and by reducing the content of the brilliant powder as much as possible.

Hereinafter, each component of the present invention will be described.
(Component (A); non-brilliant powder in which (a-2) a plate-like powder is coated with (a-1) a chromatic iron oxide)

The component (A) in the present invention is a non-brilliant powder in which (a-2) a plate-like powder is coated with (a-1) a chromatic iron oxide.

The non-brilliant powder to be used in the present invention refers to a powder having no brightness, and the presence or absence of brightness is measured by the following colorimetry method. Using a gloss meter GlossMeter VG7000 (manufactured by NIPPON DENSHOKU INDUSTRIES CO., LTD.), 10 mg of a brilliant powder was uniformly applied to a black artificial skin membrane of 10 cm long x 6 cm wide to prepare a powder coating film, the powder coating film was placed on a sample stage, and the glossiness at an incident angle of 60° and a measurement angle of 60° was measured, and defined as follows.
Glossiness of 3 or more at a measurement angle of 60°: there is brightness
Glossiness of less than 3 at a measurement angle of 60°: there is no brightness
The non-brilliant powder preferably has a glossiness of 0 to 2.5.

The (a-1) is not particularly limited as long as it is a chromatic iron oxide usually used for cosmetics. The chromatic color refers to a color having saturation, and preferably refers to a color having hue, lightness, and saturation. Having saturation refers to a color whose saturation is not substantially zero (0). As the saturation, for example, a colorimetric value C* (saturation) of a color value defined in the CIE1976L*a*b* color system can be measured by a spectral colorimeter SE7700 (manufactured by NIPPON DENSHOKU INDUSTRIES CO., LTD.). Examples of the chromatic iron oxide include red iron oxide (Bengara), yellow iron oxide, and the like.

The component (A) may be an aspect in which the (a-2) plate-like powder is coated only with (a-1) (that is, the coating powder is only the component (a-1)). The component (A) may be an aspect composed of the (a-2) plate-like powder and the (a-1) chromatic iron oxide. With such an aspect, the effect of the present invention such as absence of an appearance color difference before and after use is easily obtained, which is preferable.

The (a-2) is a plate-like powder. The plate-like powder refers to a powder having an average particle diameter larger than an average thickness, and specifically, the aspect ratio is preferably 3 or more, and may be 3 to 1000 or 5 to 100. The aspect ratio is calculated by a ratio between the average particle diameter and the average thickness of the particles, and is defined by aspect ratio=(average particle diameter/average thickness). The average particle diameter is calculated from the result of observing the particle diameters (maximum diameters) of 10 particles in an arbitrary field of view using a scanning electron microscope. Similarly, the particle thickness is obtained, and the particle diameter is divided by the thickness to calculate the aspect ratio.

The average particle diameter of (a-2) is, for example, 3 to 30 μm.

The (a-2) is not particularly limited as long as it is a plate-like powder usually used for cosmetics, examples thereof include extender powders such as mica, sericite, synthetic phlogopite, synthetic sericite, aluminum silicate, magnesium silicate, magnesium aluminum silicate, cleavage talc, plate-like anhydrous silicic acid, plate-like aluminum oxide, plate-like kaolin, plate-like boron nitride, plate-like titanium oxide, and plate-like cellulose, resin laminated powders such as titanium mica, bismuth oxychloride, fish scale foil, polyethylene terephthalate-aluminum-epoxy laminated powder, polyethylene terephthalate-polyolefin laminated film powder, and polyethylene terephthalate-polymethyl methacrylate laminated film powder, and the like, and one or two or more kinds thereof can be used in combination. Among them, the (a-2) is preferably mica and/or synthetic phlogopite, and more preferably synthetic phlogopite. The powder used in the present application is a “non-brilliant powder” in which the (a-2) plate-like powder is coated with the (a-1) chromatic iron oxide, and is distinguished from iron oxide-coated synthetic phlogopite or the like known as a so-called pearl agent (brilliant powder). The component (A) of the present invention is a powder in which the (a-2) plate-like powder is coated with the (a-1) chromatic iron oxide so as to be non-brilliant.

When the component (A) is non-brilliant, the (a-2) may be brilliant, but the (a-2) is also preferably non-brilliant.

The coating amount of the (a-1) with respect to the total amount of the component (A) is not particularly limited, but is preferably 10 to 80% and more preferably 15 to 60%.

In a method of coating the plate-like powder with iron oxide, it is necessary to perform coating so that a powder to be obtained becomes non-brilliant. For example, by coating the (a-2) plate-like powder with the (a-1) chromatic iron oxide having a relatively large particle diameter (for example, the average particle diameter is 40 to 400 nm, preferably 70 to 250 nm, and more preferably more than 100 nm and 250 nm or less), non-brilliance can be achieved by the scattering effect of light due to the coating with the (a-1). Examples of the coating with the iron oxide include a method of adding a water-soluble metal salt such as ferric chloride to an aqueous suspension of a plate-like powder, neutralizing and decomposing the mixture to precipitate hydrated iron oxide, and firing the mixture; a method of spray-drying a dispersion of a plate-like powder and an iron oxide powder using an electrostatic force of the plate-like powder and the iron oxide powder; and the like. By these methods, a non-brilliant powder can be obtained by appropriately setting reaction conditions (reaction time, reaction temperature, and the like) so as to be non-brilliant. As the component (A), a commercially available product may be used, and examples of the commercially available product include NK-Red and NK-Yellow (both manufactured by NIHON KOKEN KOGYO CO., LTD.), and the like.

As the component (A), since the absence of an appearance color difference before and after use is further exhibited, an embodiment in which red iron oxide-coated synthetic phlogopite and yellow iron oxide-coated synthetic phlogopite are used in combination is also suitable. In the case of using red iron oxide-coated synthetic phlogopite and yellow iron oxide-coated synthetic phlogopite in combination, the blending mass ratio of both components is, for example, the red iron oxide-coated synthetic phlogopite: the yellow iron oxide-coated synthetic phlogopite=1:0.1 to 1:10, and may be 1:0.5 to 1:5.

The average particle diameter of the component (A) is, for example, 3 to 30 μm.

The content of the component (A) (the total amount when there are two or more kinds of the component (A)) in the cosmetic is not particularly limited, and the lower limit value is preferably 5% or more, more preferably 10% or more, further preferably 15% or more, still more preferably 20% or more, and particularly preferably 25% or more, from the viewpoint of absence of an appearance color difference, moldability, easiness of scooping, and the like. The upper limit value is preferably 90% or less, more preferably 80% or less, and further preferably 70% or less, and may be 60% or less, 50% or less, or 45% or less.

### (Component (B); Non-Volatile Oil)

By blending the component (B), the moldability in a system in which the component (A) is blended is improved. In the present specification, a volatile oil agent means a liquid oil having a boiling point at normal pressure of 260° C. or lower and fluidity at normal temperature (25° C.), and the non-volatile oil refers to an oil agent other than the volatile oil agent.

The component (B) in the present invention can be used regardless of the origin of animal oils, vegetable oils, synthetic oils, and the like, and the properties of solid oils, semi-solid oils, liquid oils, and the like as long as it is a nonvolatile oil agent usually used for cosmetics, and examples thereof include hydrocarbons, fats and oils, ester oils, fatty acids, higher alcohols, silicone oils, fluorine-based oils, and lanolin derivatives. Specific examples thereof include hydrocarbons such as liquid paraffin, squalane, petrolatum, polybutene, hydrogenated polyisobutene (heavy liquid isoparaffin), paraffin wax, ceresin wax, microcrystalline wax, polyethylene wax, ethylene-propylene copolymer, montan wax, and Fischer-Tropsch wax, oils and fats such as Japan wax, olive oil, castor oil, mink oil, and macadamia nut oil, waxes such as beeswax, carnauba wax, candelilla wax, and spermaceti, ester oils such as glyceryl triethylhexanoate, cetyl isooctanate, isopropyl myristate, isopropyl palmitate, diisostearyl malate, octyldodecyl myristate, cetyl 2-ethylhexanoate, glyceryl trioctanoate, neopentyl dioctanate glycol, cholesterol fatty acid ester, dipentaerythritol fatty acid ester, N-lauroyl-L-glutamic acid di(cholesteryl behenyl octyldodecyl), and jojoba oil, fatty acids such as stearic acid, lauric acid, myristic acid, behenic acid, isostearic acid, and oleic acid, higher alcohols such as stearyl alcohol, cetyl alcohol, lauryl alcohol, oleyl alcohol, isostearyl alcohol, and behenyl alcohol, silicones such as dimethylpolysiloxane (with a high polymerization degree) (dimethicone, methylpolysiloxane), methylphenylpolysiloxane, partially crosslinked organopolysiloxane, and fluorine-modified polysiloxane, fluorine-based oil agents such as perfluorodecane, perfluorooctane, and perfluoropolysiloxane, lanolin derivatives such as lanolin, lanolin acetate, isopropyl lanolin fatty acid, and lanolin alcohol, oily gelling agents such as dextrin fatty acid ester, sucrose fatty acid ester, 12-hydroxystearic acid ester, starch fatty acid ester, aluminum isostearate, and calcium stearate, and the like. The component (B) may be used singly or in combination of two or more kinds thereof. Among them, from the viewpoint of moldability, easiness of scooping, and the like, the component (B) is preferably at least one selected from the group consisting of hydrocarbon oil, ester oil, and silicone oil, more preferably includes at least ester oil and/or silicone oil, and from the viewpoint of moldability and easiness of scooping, the component (B) further preferably includes at least ester oil and silicone oil, still more preferably include silicone oil and diisostearyl malate, and particularly preferably includes silicone oil having a kinematic viscosity at 25° C. of 8 mm²/s or more and less than 20 mm²/s and diisostearyl malate. The mass content ratio in the case of containing ester oil and silicone oil is, for example, 1:0.1 to 1:10 and may be 1:0.5 to 1:5.

The ester oil is not particularly limited, but from the viewpoint of moldability and the like, preferably contains at least one selected from the group consisting of glyceryl triethylhexanoate, cetyl isooctanate, isopropyl myristate, isopropyl palmitate, diisostearyl malate, octyldodecyl myristate, cetyl 2-ethylhexanoate, glyceryl trioctanoate, and neopentyl glycol dioctanoate, and more preferably contains diisostearyl malate and/or cetyl 2-ethylhexanoate.

The kinematic viscosity of the silicone oil at 25° C. is not particularly limited, and is preferably 4 to 50 mm²/s, more preferably 6 to 30 mm²/s, and further preferably 8 to 25 mm²/s. From the viewpoint of moldability and easiness of scooping, the component (B) further preferably contains at least a silicone oil having a kinematic viscosity at 25° C. of 8 mm²/s or more and less than 20 mm²/s. The silicone oil preferably contains at least dimethylpolysiloxane, and may be only dimethylpolysiloxane.

The content of the component (B) (the total amount when there are two or more kinds of the component (B)) in the cosmetic is not particularly limited, and the lower limit value is, for example, 3% or more and may be 5% or more, and from the viewpoint of moldability, easiness of scooping, and the like, the lower limit value is preferably 15% or more and more preferably 18% or more, and the upper limit value is preferably 40% or less and more preferably 30% or less. The content of the component (B) in the cosmetic is preferably 15 to 40% and more preferably 18 to 30%. As the component (B), an oil agent used as a surface treatment agent for powders is not included.

The solid powder cosmetic of the present invention does not contain a chromatic iron oxide not coating the powder component or the content of the iron oxide (the total amount when there are two or more kinds of the iron oxide) in the cosmetic is 15% or less. When a chromatic iron oxide not coating the powder component is not contained or the content of the iron oxide in the cosmetic is 15% or less, the effect is exerted in terms of absence of an appearance color difference before and after use and moldability. It is preferable that the solid powder cosmetic does not contain a chromatic iron oxide not coating the powder component or the content of the iron oxide in the cosmetic is 10 mass % or less, 5 mass % or less, 3 mass % or less, 1 mass % or less, 0.5 mass % or less, or 0.1 mass % or less.

The chromatic iron oxide not coating the powder component in the solid powder cosmetic of the present invention refers to an iron oxide to be blended in the cosmetic without coating the powder component. The powder component in the chromatic iron oxide not coating the powder component is not limited to the (a-2) plate-like powder. The chromatic iron oxide not coating the powder component preferably refers to a chromatic iron oxide simple substance (that is, a composite powder is not formed with another powder), and more preferably red iron oxide and/or yellow iron oxide. It is preferable that the iron oxide is not contained or the content of the iron oxide in the cosmetic is 5% or less, and it is more preferable that the iron oxide is not contained. One aspect of the solid powder cosmetic of the present invention does not contain red iron oxide and/or yellow iron oxide (provided that the component (A) is excluded) or the content of the iron oxide is 15 mass % or less, 10 mass % or less, 5 mass % or less, 3 mass % or less, 1 mass % or less, 0.5 mass % or less, or 0.1 mass % or less with respect to the total amount of the cosmetic.

One preferred aspect of the present invention does not contain an iron oxide not coating the powder component or the content of the iron oxide in the cosmetic is 15% or less. Such an aspect is more unlikely to cause a color difference in appearance. The iron oxide not coating the powder component in the solid powder cosmetic of the present invention refers to an iron oxide to be blended in the cosmetic without coating the powder component. The powder component in the iron oxide not coating the powder component is not limited to the (a-2) plate-like powder. The iron oxide not coating the powder component preferably refers to an iron oxide simple substance (that is, a composite powder is not formed with another powder). It is preferable that the iron oxide is not contained or the content of the iron oxide in the cosmetic is 5% or less, and it is more preferable that the iron oxide is not contained. One aspect of the solid powder cosmetic of the present invention does not contain any of a red iron oxide simple substance, a yellow iron oxide simple substance, and a black iron oxide simple substance, or the content (total amount) of the iron oxide(s) is 15 mass % or less, 10 mass % or less, 5 mass % or less, 3 mass % or less, 1 mass % or less, 0.5 mass % or less, or 0.1 mass % or less with respect to the total amount of the cosmetic.

In the solid powder cosmetic of the present invention, the mass content ratio of the (a-1) and the chromatic iron oxide not coating the powder component, i.e., (a-1):(the chromatic iron oxide not coating the powder component) is 100:0 to 20:80, and is more preferably 100:0 to 50:50, 100:0 to 60:40, 100:0 to 70:30, 100:0 to 80:20, or 100:0 to 90:10 from the viewpoint of absence of an appearance color difference, moldability, easiness of scooping, and the like.

The solid powder cosmetic of the present invention does not contain a brilliant powder or the content of the brilliant powder in the cosmetic is 15 mass % or less. From the viewpoint of absence of an appearance color difference, moldability, easiness of scooping, and the like, it is preferable that a brilliant powder is not contained or the content of the brilliant powder in the cosmetic is 5 mass % or less, 3 mass % or less, 1 mass % or less, 0.5 mass % or less, or 0.1 mass % or less, and it is more preferable that the brilliant powder is not contained (the content of the brilliant powder is 0 mass %). The brilliant powder refers to a powder having brightness, and specifically refers to a powder that there is brightness measured by the colorimetry method described above.

The brilliant powder is not particularly limited as long as it is a powder having brightness, and specific examples thereof include titanium oxide-coated mica, titanium oxide-coated synthetic phlogopite, black iron oxide-coated mica titanium, iron oxide-black iron oxide-coated mica titanium, indigo blue-coated mica titanium, carmine-coated mica titanium, carmine-indigo blue-coated mica titanium, iron oxide-carmine-coated mica titanium, iron oxide-indigo blue-coated mica titanium, organic pigment-coated mica titanium, metal powder-coated mica titanium, metal powder-coated synthetic phlogopite, glass powder, titanium oxide-coated glass flakes, metal powder-coated glass powder, aluminum flakes, titanium oxide-coated alumina flakes, titanium oxide-coated silica flakes, iron oxide-silica-coated aluminum, polyethylene terephthalate-aluminum-epoxy laminated powder, polyethylene terephthalate-polyolefin laminated film powder, polyethylene terephthalate-polymethyl methacrylate laminated film powder, goniochromatic gloss pigment, fish scale foil, gold powder, borosilicate (Ca/Al)-titanium oxide-tin oxide, synthetic phlogopite-titanium oxide-tin oxide, silver powder, and the like.

In the solid powder cosmetic of the present invention, black iron oxide may be used as color adjustment. In the case of combining black iron oxide, it is preferable to use a black iron oxide simple substance (that is, it is not a composite powder with another powder).

As the solid powder cosmetic of the present invention, an aspect containing a surfactant is preferable. The surfactant may be any surfactant that is usually used in cosmetics, and examples thereof include a nonionic surfactant, an anionic surfactant, a cationic surfactant, an amphoteric surfactant, and the like. Among them, a nonionic surfactant is preferably used from the viewpoint of the effect of the present invention. Examples of the nonionic surfactant include glycerin fatty acid ester, sorbitan fatty acid ester, sucrose fatty acid ester, fatty alcohol ethoxylate, polyoxyethylene alkyl phenyl ether, alkyl glycoside, and the like, and one or two or more of these can be used. Among them, it is preferable to use a nonionic surfactant having HLB of 3 or more and 7 or less or 3 or more and 6 or less. Examples of such a surfactant include glyceryl myristate (HLB value 3.5), glyceryl monostearate (HLB value 4.0), sorbitan sesquistearate (HLB 4.0), sorbitan sesquiisostearate (HLB 4.0), sorbitan monooleate (HLB 4.3), diglyceryl monostearate (HLB value 5.0), diglyceryl monooleate (HLB value 6.5), diglyceryl dioleate (HLB value 7.0), diglyceryl monoisostearate (HLB value 5.5), tetraglyceryl monostearate (HLB value 6.0), tetraglyceryl monooleate (HLB value 6.0), POE (5) glyceryl triisostearate (HLB value 3.0), POE (10) glyceryl triisostearate (HLB value 5.0), POE (20) glyceryl triisostearate (HLB value 8.0), POE (5) decyl tetradecyl ether (HLB value 6.0), POE (5) cholesteryl ether (HLB value 7.0), and the like.

The solid powder cosmetic of the present invention can appropriately contain various components other than the essential components, for example, a powder other than the component (A), a surfactant, an aqueous component, a water-soluble polymer, a solid ultraviolet absorber, a moisturizing agent, a fading inhibitor, an antioxidant, an antifoaming agent, a cosmetic component, a preservative, a fragrance, and the like in order to impart various effects, as necessary, to the extent that the effects of the present invention are not impaired. Although not particularly limited, talc, sericite, and surface-treated powders thereof can be used as an additional material (other than the component (A) and the like, a material other than a functional material that imparts a cosmetic effect) in the solid powder cosmetic. These additional materials are, for example, 20 to 90% and may be 30 to 80% in the solid powder cosmetic.

The aqueous component may be any component as long as it is water and a component soluble in water, and examples thereof include glycols such as propylene glycol, 1,3-butylene glycol, dipropylene glycol, and polyethylene glycol, glycerols such as glycerin, diglycerin, and polyglycerin, plant extracts such as aloe vera, witch hazel, hamamelis, cucumber, lemon, lavender, and rose, and the like. However, in the present invention, in order not to impair the obtained effect, the content of the aqueous component is more preferably 20% or less, 15% or less, 10% or less, 5% or less, 3% or less, or 1% or less in the solid powder cosmetic.

Examples of the water-soluble polymer include natural polymers such as guar gum, sodium chondroitin sulfate, Na hyaluronate, gum arabic, sodium alginate, carrageenan, mucopolysaccharide, collagen, elastin, and keratin, semisynthetic polymers such as methyl cellulose, hydroxyethyl cellulose, and carboxymethyl cellulose, and synthetic polymers such as carboxyvinyl polymer, polyvinyl alcohol, polyvinyl pyrrolidone, and sodium polyacrylate.

Examples of the solid ultraviolet absorber include benzophenone-based absorber, PABA-based absorber, 4-tert-butyl-4′-methoxydibenzoylmethane, oxybenzone, and the like. Examples of the antioxidant include a-tocopherol, ascorbic acid, and the like, examples of the cosmetic component include vitamins, proteins, antiinflammatory agents, crude drugs, and the like, and examples of the preservative include paraoxybenzoic acid ester, phenoxyethanol, and the like.

Examples of the solid powder cosmetic of the present invention include makeup cosmetics such as foundation, blusher, lipstick, eye shadow, eyebrow, and concealer, skin care cosmetics such as sunscreen cosmetics, and the like, and cosmetics in which the effect of the present invention is remarkably exhibited are makeup cosmetics such as foundation, eye shadow, and blusher.

The solid powder cosmetic of the present invention can be obtained by adding a solvent to a cosmetic base obtained by mixing the component (A), the component (B), and as necessary, other components, dispersing the mixture to form a slurry, filling the slurry in a container, and then removing the solvent. A method of mixing the cosmetic base is not particularly limited, and a generally known method may be used. As a method of removing the solvent (molding method), a generally known method such as a method of filling a cosmetic base in a slurry form, then pressurizing the cosmetic base to remove an excessive solvent, and then drying the cosmetic base, or a generally known method such as a method of removing the solvent using an absorber such as paper or a nonwoven fabric or through a discharge hole can be used. Examples of the solvent include aqueous components such as water and ethanol, and a volatile oil agent. Among them, the solvent is preferably a volatile oil agent from the viewpoint of moldability and easiness of scooping, and for example, as the volatile oil agent, light isoparaffin, isododecane, isohexadecane, cyclic silicone, and the like can be used. The amount of the solvent to be added is preferably 15 to 40 parts by mass and more preferably 20 to 30 parts by mass with respect to 100 parts by mass of the cosmetic base.

Since the solvent is basically volatilized by 100%, the content of the component in the solid powder cosmetic coincides with the content of the component in the cosmetic base.

In a wet production method (a method of adding a solvent to a cosmetic base, dispersing the mixture to form a slurry, filling the slurry in a container, and then removing the solvent), the container is filled with the flowable cosmetic base in a slurry state, and the cosmetic base flows during filling, so that the plate-like powder tends to be oriented in the flow direction. In particular, the plate-like powder near the surface strongly tends to be oriented parallel to the surface, and the solid powder cosmetic molded by the wet production method has a state in which the plate-like powder is regularly arranged. In the solid powder cosmetic molded by a dry method, it is considered that the direction of the plate-like powder is random. Therefore, the structure of a cosmetic to be obtained is also different in each molding method. Since the orientation of the powder and the localization of the oil agent are different depending on the solvent to be used, the structure of a cosmetic to be obtained is different.

The present invention can also adopt the following configurations.

<1>

A solid powder cosmetic being obtained by adding a solvent to a cosmetic base containing the following components (A) and (B);
(A) a non-brilliant powder in which (a-2) a plate-like powder is coated with (a-1) a chromatic iron oxide, and
(B) a non-volatile oil
to form a slurry, filling a container with the slurry, and then removing the solvent,
in which a chromatic iron oxide not coating a powder component is not contained or a content of the iron oxide is 15% or less with respect to a total amount of the cosmetic,
a brilliant powder is not contained or a content of the brilliant powder is 15% or less with respect to the total amount of the cosmetic, and
a mass content ratio of the (a-1) and the chromatic iron oxide not coating the powder component, i.e., (a-1):(the chromatic iron oxide not coating the powder component) is 100:0 to 20:80.
   <2>

The solid powder cosmetic according to <1>, wherein the (a-2) is one or two or more kinds selected from mica and synthetic phlogopite.

<3>

The solid powder cosmetic according to <1> or <2>, wherein the brilliant powder is not substantially contained or the content of the brilliant powder with respect to the total amount of the cosmetic is 5% or less.

<4>

The solid powder cosmetic according to any one of <1> to <3>, wherein the component (B) is one or two or more kinds selected from the group consisting of a hydrocarbon oil, a silicone oil, and an ester oil.

<5>

The solid powder cosmetic according to <4>, wherein the ester oil contains malic acid diester and/or cetyl 2-ethylhexanoate.

<6>

The solid powder cosmetic according to any one of <1> to <5>, wherein the component (A) is a combination of red iron oxide-coated synthetic phlogopite and yellow iron oxide-coated synthetic phlogopite.

### EXAMPLES

Hereinafter, the present invention will be described in detail with reference to Examples. These do not limit the present invention at all.

### Examples 1 to 14 and Comparative Examples 1 to 5: Solid Powder Cosmetic (Eye Shadow)

A solid powder cosmetic having a formulation shown in the following Tables 1 and 2 was prepared according to the following production method, and evaluated for each item of (1) absence of an appearance color difference before and after use, (2) moldability, (3) easiness of scooping, and (4) matte texture by the following evaluation methods. The results are also shown in Tables 1 and 2.

TABLE 1
(%)

| | | | Example | | | | | | | | |
|---|---|---|---|---|---|---|---|---|---|---|---|
| Classification | No | Component name | 1 | 2 | 3 | 4 | 5 | 6 | 7 | 8 | 9 |
| (A) | 1 | Red iron oxide-coated | 7 | 4 | 1.5 | 3.5 | 7 | 7 | 7 | 7 | 7 |
| | | plate-like synthetic | | | | | | | | | |
| | | phlogopite ※1 | | | | | | | | | |
| | 2 | Yellow iron oxide-coated | 20 | 10 | 4 | 10 | 20 | 20 | 20 | 20 | 20 |
| | | plate-like synthetic | | | | | | | | | |
| | | phlogopite ※2 | | | | | | | | | |
| | 3 | Mica | — | — | — | — | — | — | — | — | — |
| Chromatic iron | 4 | Red iron oxide | — | — | — | 5 | — | — | — | — | — |
| oxide not | 5 | Yellow iron oxide | — | — | — | 10 | — | — | — | — | — |
| coating powder | | | | | | | | | | | |
| component | | | | | | | | | | | |
| Brilliant powder | 6 | Borosilicic acid | — | — | — | — | 5 | 15 | — | — | — |
| | | (Ca/Al)/titanium oxide/tin | | | | | | | | | |
| | | oxide ※3 | | | | | | | | | |
| | 7 | Synthetic | — | — | — | — | — | — | 5 | 15 | — |
| | | phlogopite/titanium oxide/tin | | | | | | | | | |
| | | oxide ※4 | | | | | | | | | |
| (B) | 8 | Dimethicone (10 mm²/s) | 10 | 10 | 10 | 10 | 10 | 10 | 10 | 10 | — |
| | 9 | Dimethicone (20 mm²/s) | — | — | — | — | — | — | — | — | 10 |
| | 10 | Dimethicone (6 mm²/s) | — | — | — | — | — | — | — | — | — |
| | 11 | Diisostearyl malate | 10 | 10 | 10 | 10 | 10 | 10 | 10 | 10 | 10 |
| | 12 | Glyceryl triethylhexanoate | — | — | — | — | — | — | — | — | — |
| Arbitrary | 13 | Sorbitan sesquistearate | 2 | 2 | 2 | 2 | 2 | 2 | 2 | 2 | 2 |
| component | 14 | Talc | Balance | Balance | Balance | Balance | Balance | Balance | Balance | Balance | Balance |
| | | (a-1):(chromatic iron oxide | 100:0 | 100:0 | 100:0 | 20:80 | 100:0 | 100:0 | 100:0 | 100:0 | 100:0 |
| | | not coating powder | | | | | | | | | |
| | | component) | | | | | | | | | |
| | | Solvent | Isododecane | Isododecane | Isododecane | Isododecane | Isododecane | Isododecane | Isododecane | Isododecane | Isododecane |
| | <Items and determination results> | | | | | | | | | | |
| | (1) | Absence of appearance | ⊙ | ⊙ | ◯ | ◯ | ⊙ | ◯ | ⊙ | ◯ | ⊙ |
| | | color difference before and | | | | | | | | | |
| | | after use | | | | | | | | | |
| | (2) | Moldability | ⊙ | ⊙ | ◯ | ◯ | ⊙ | ⊙ | ⊙ | ⊙ | ◯ |
| | (3) | Easiness of scooping | ⊙ | ◯ | ◯ | ◯ | ◯ | ◯ | ◯ | ◯ | ◯ |
| | (4) | Matte texture | ⊙ | ⊙ | ⊙ | ⊙ | ◯ | ◯ | ◯ | ◯ | ⊙ |

※1 Red iron oxide coating amount 50%
※2 Yellow iron oxide coating amount 20%
※3 METASHINE 1080RR (manufactured by Nippon Sheet Glass Co., Ltd.)
※4 TWINCLE PEARL 410 (manufactured by NIHON KOKEN KOGYO CO., LTD.)

TABLE 2
(%)

| | | | Example | | | | | Comparative Example | | | | |
|---|---|---|---|---|---|---|---|---|---|---|---|---|
| | No | Component name | 10 | 11 | 12 | 13 | 14 | 1 | 2 | 3 | 4 | 5 |
| (A) | 1 | Red iron oxide-coated | 7 | 7 | 7 | 7 | 7 | 2.4 | — | 7 | 4 | 4 |
| | | plate-like synthetic | | | | | | | | | | |
| | | phlogopite ※1 | | | | | | | | | | |
| | 2 | Yellow iron oxide-coated | 20 | 20 | 20 | 20 | 20 | 8 | — | 20 | 10 | 10 |
| | | plate-like synthetic | | | | | | | | | | |
| | | phlogopite ※2 | | | | | | | | | | |
| | 3 | Mica | — | — | — | — | — | — | 13.5 | — | — | — |
| Chromatic iron | 4 | Red iron oxide | — | — | — | — | — | 2 | 3.5 | — | 8 | 8 |
| oxide not | 5 | Yellow iron oxide | — | — | — | — | — | 16 | 10 | — | 8 | 8 |
| coating powder | | | | | | | | | | | | |
| component | | | | | | | | | | | | |
| Brilliant | 6 | Borosilicic acid | — | — | — | — | — | — | — | — | — | — |
| powder | | (Ca/Al)/titanium oxide/tin | | | | | | | | | | |
| | | oxide ※3 | | | | | | | | | | |
| | 7 | Synthetic | — | — | — | — | — | — | — | 20 | — | — |
| | | phlogopite/titanium | | | | | | | | | | |
| | | oxide/tin oxide ※4 | | | | | | | | | | |
| (B) | 8 | Dimethicone (10 mm²/s) | — | — | 20 | — | 10 | 10 | 10 | 10 | 10 | — |
| | 9 | Dimethicone (20 mm²/s) | — | — | — | — | — | — | — | — | — | — |
| | 10 | Dimethicone (6 mm²/s) | 10 | — | — | — | — | — | — | — | — | — |
| | 11 | Diisostearyl malate | 10 | — | — | 20 | 10 | 10 | 10 | 10 | 10 | — |
| | 12 | Glyceryl triethylhexanoate | — | 20 | — | — | — | — | — | — | — | — |
| Arbitrary | 13 | Sorbitan sesquistearate | 2 | 2 | 2 | 2 | 2 | 2 | 2 | 2 | 2 | 2 |
| | 14 | Talc | Balance | Balance | Balance | Balance | Balance | Balance | Balance | Balance | Balance | Balance |
| | | (a-1):(chromatic iron | 100:0 | 100:0 | 100:0 | 100:0 | 100:0 | 10:90 | 0:100 | 100:0 | 20:80 | 20:80 |
| | | oxide not coating | | | | | | | | | | |
| | | powder component) | | | | | | | | | | |
| | | Solvent | Isododecane | Isododecane | Isododecane | Isododecane | Isododecane | Isododecane | Isododecane | Isododecane | Isododecane | Isododecane |
| | <Items and determination results> | | | | | | | | | | | |
| | (1) | Absence of appearance | ⊙ | ⊙ | ⊙ | ⊙ | ⊙ | Δ | X | Δ | X | ◯ |
| | | color difference before and | | | | | | | | | | |
| | | after use | | | | | | | | | | |
| | (2) | Moldability | ◯ | ◯ | ◯ | ◯ | ◯ | ◯ | Δ | Δ | Δ | X |
| | (3) | Easiness of scooping | ◯ | ◯ | ◯ | ◯ | ◯ | ◯ | ◯ | ◯ | ◯ | Δ |
| | (4) | Matte texture | ⊙ | ⊙ | ⊙ | ⊙ | ⊙ | ⊙ | ⊙ | X | ◯ | ⊙ |

### (Production Method)

A. Components (1) to (7) and (14) were uniformly mixed.
B. Components (8) to (13) were uniformly mixed.
C. B was added to A to obtain a cosmetic base, 20 parts by mass of isododecane or water was added to 100 parts by mass of the cosmetic base, mixing and kneading were performed, and 2.5 g of the mixture was filled in a metal container (2.5 cm×2 cm×0.3 cm).
D. Two sheets of cellulose paper having a thickness of 0.02 mm were stacked on the surface of C, isododecane or water was removed by suction for 4 seconds while compression molding was performed at 2 kgf/cm² using a press mold from above, and then drying was performed at 70° C. for 8 hours to obtain a solid powder cosmetic (eye shadow).

### (Evaluation Item)

(1) Absence of appearance color difference before and after use

### (Evaluation Method)

For each sample, the color of the sample surface before use and the color of the sample surface after 10 times of rubbing the cosmetic surface using a cosmetic chip were each measured with a spectral colorimeter SE-2000 (NIPPON DENSHOKU INDUSTRIES CO., LTD.), calculated by a ΔE value of a Hunter color system, and then determined according to the following four-stage criteria for determination.

### (Four-Stage Criteria for Determination)

### (Determination): (Criteria for Determination)

⊙: ΔE is less than 3
∘: ΔE is 3 or more and less than 4
Δ: ΔΕ is 4 or more and less than 4.5
x: ΔE is 4.5 or more

### (Evaluation Item)

(2) Moldability

### (Evaluation Method)

For each sample before drying from which the solvent was removed by suction, the penetration hardness (g/cm²) was measured under the measurement conditions (needle: 2 mm, speed: 2 cm/min, depth: 1 mm) using a rheometer (FUDOH rheometer manufactured by Rheotech Co., Ltd.), and an average value of the number of tests (N)=3 was calculated and determined according to the following four-stage criteria for determination.

### (Four-Stage Criteria for Determination)

### (Determination): (Criteria for Determination)

⊙: 200 g/cm² or more
∘: 150 g/cm² or more and less than 200 g/cm²
Δ: 100 g/cm² or more and less than 150 g/cm²
x: less than 100 g/cm²

### (Evaluation Item)

(3) Easiness of scooping
(4) Matte texture

### (Evaluation Method)

Each sample was subjected to a use test by 20 cosmetic evaluation specialized panels, and each of the items was evaluated in five stages according to the following evaluation criteria and rated, an average score of the scores of all the panels was calculated for each sample, and the samples were determined according to the following criteria.

As for (3) easiness of scooping, whether or not the amount of the sample attached to the finger when the sample was taken with the finger was appropriate was evaluated.

As for (4) matte texture, whether or not the appearance of the sample surface was matte was visually evaluated.

### (Evaluation Criteria)

### (Score): (Evaluation)

5: Very good
4: Good
3: Normal
2: Slightly poor
1: Poor

### (Criteria for Determination)

(Determination): (Average score of scores)
⊙: 4.0 or more
∘: 3.5 points or more and less than 4.0 points
Δ: 2.0 points or more and less than 3.5 points
x: less than 2.0 points

As is clear from the results of Tables 1 and 2, the solid powder cosmetics of Examples 1 to 14 were excellent in all items. In Examples 1 to 14, the measured values ΔE of the appearance color difference before and after use were all 3.5 or less. On the other hand, in Comparative Example 1 in which the amount of the chromatic iron oxide not coating the powder component was larger than the amount of (a-1) in the component (A), a satisfactory result was not obtained in the appearance color difference. In Comparative Example 2 containing no component (A), satisfactory results were not obtained in the appearance color difference and the moldability. In Comparative Example 3 in which the content of the brilliant powder was more than 15%, satisfactory results were not obtained in the moldability and the matte texture. In Comparative Example 4 in which the content of the chromatic iron oxide not coating the powder component was more than 15%, satisfactory results were not obtained in the appearance color difference and the moldability.

### Example 15: Solid Powder Cosmetic (Blusher)

A solid powder cosmetic (blusher) was produced by the following formulation and production method.

| (Component) | (%) |
|---|---|
| 1. Mica | 25 |
| 2. Synthetic phlogopite | 10 |
| 3. Silicon-treated anhydrous silicic acid/mica | 8 |
| 4. Alkyl polyacrylate | 5 |
| 5. Zinc oxide | 5 |
| 6. Red iron oxide-coated synthetic phlogopite ※1 | 8 |
| (component (A)) | |
| 7. Yellow iron oxide-coated synthetic phlogopite ※2 | 15 |
| (component (A)) | |
| 8. Talc balance | balance |
| 9. Petrolatum (component (B)) | 4 |
| 10. Diisostearyl malate (component (B)) | 2 |
| 11. Ethanol | 0.5 |
| 12. Plant-derived squalane (component (B)) | 0.2 |
| 13. Fragrance | 1 |

(Production method)
A. Components (1) to (8) were uniformly mixed.
B. Components (9) to (13) were uniformly mixed.

C. B was added to A to obtain a cosmetic base, 20 parts by mass of isododecane was added to 100 parts by mass of the cosmetic base, mixing and kneading were performed, and 2.5 g of the mixture was filled in a metal container (2.5 cm×2 cm×0.3 cm).

D. Two sheets of cellulose paper having a thickness of 0.02 mm were stacked on the surface of C, isododecane was removed by suction for 4 seconds while compression molding was performed at 2 kgf/cm² using a press mold from above, and then drying was performed at 70° C. for 8 hours to obtain a solid powder cosmetic (blusher).

The solid powder cosmetic (blusher) obtained as described above was satisfactory in all respects of absence of an appearance color difference, moldability, easiness of scooping, and matte texture.

### Example 16: Solid Powder Cosmetic (Eye Shadow)

A solid powder cosmetic (eye shadow) was produced by the following formulation and production method.

| (Component) | (%) |
|---|---|
| 1. Talc | balance |
| 2. Zinc oxide | 5 |
| 3. Borosilicic acid (Ca/Al)/titanium oxide/tin oxide | 2 |
| (brilliant powder) | |
| 4. Red iron oxide-coated synthetic phlogopite ※1 | 10 |
| (component (A)) | |
| 5. Yellow iron oxide-coated synthetic phlogopite ※2 | 8 |
| (component (A)) | |
| 6. Sorbitan sesquistearate | 5 |
| 7. Heavy liquid isoparaffin (component (B) | 10 |
| 8. Liquid paraffin (component (B)) | 10 |

(Production method)
A. Components (1) to (5) were uniformly mixed.
B. Components (6) to (8) were uniformly mixed.

C. B was added to A to obtain a cosmetic base, 20 parts by mass of isododecane was added to 100 parts by mass of the cosmetic base, mixing and kneading were performed, and 2.5 g of the mixture was filled in a metal container (2.5 cm×2 cm×0.3 cm).

D. Two sheets of cellulose paper having a thickness of 0.02 mm were stacked on the surface of C, isododecane was removed by suction for 4 seconds while compression molding was performed at 2 kgf/cm² using a press mold from above, and then drying was performed at 70° C. for 8 hours to obtain a solid powder cosmetic (eye shadow).

The solid powder cosmetic (eye shadow) obtained as described above was satisfactory in all respects of absence of an appearance color difference, moldability, easiness of scooping, and matte texture.

### Example 17: Solid Powder Cosmetic (Eye Shadow)

A solid powder cosmetic (eye shadow) was produced by the following formulation and production method.

| (Component) | (%) |
|---|---|
| 1. Talc | balance |
| 2. Mica | 10 |
| 3. Methyl methacrylate crosspolymer | 5 |
| 4. Zinc oxide | 5 |
| 5. Nylon powder | 2 |
| 6. Boron nitride | 2 |
| 7. Red iron oxide-coated synthetic phlogopite ※1 | 10 |
| (component (A)) | |
| 8. Yellow iron oxide-coated synthetic phlogopite ※2 | 8 |
| (component (A)) | |
| 9. Diisostearyl malate (component (B) | 15 |
| 10. Cetyl 2-ethylhexanoate (component (B | 3 |
| 11. Dipentaerythritol fatty acid ester (component (B)) | 1 |

(Production method)
A. Components (1) to (8) were uniformly mixed.
B. Components (9) to (11) were uniformly mixed.

C. B was added to A to obtain a cosmetic base, 20 parts by mass of isododecane was added to 100 parts by mass of the cosmetic base, mixing and kneading were performed, and 2.5 g of the mixture was filled in a metal container (2.5 cm×2 cm×0.3 cm).

D. Two sheets of cellulose paper having a thickness of 0.02 mm were stacked on the surface of C, isododecane was removed by suction for 4 seconds while compression molding was performed at 2 kgf/cm² using a press mold from above, and then drying was performed at 70° C. for 8 hours to obtain a solid powder cosmetic (eye shadow).

The solid powder cosmetic (eye shadow) obtained as described above was satisfactory in all respects of absence of an appearance color difference, moldability, easiness of scooping, and matte texture.

### Example 18: Solid Powder Cosmetic (Foundation)

A solid powder cosmetic (foundation) was produced by the following formulation and production method.

| (Component) | (%) |
|---|---|
| 1. Sericite | balance |
| 2. Mica | 10 |
| 3. Barium sulfate | 5 |
| 4. Methylpolysiloxane (6 mm²/s) (component (B)) | 3 |
| 5. Dipropylene glycol | 2 |
| 6. Methylphenylpolysiloxane (component (B) | 1 |
| 7. Hydrogenated soybean phospholipid | 0.5 |
| 8. Red iron oxide-coated synthetic phlogopite ※1 | 20 |
| (component (A)) | |
| 9. Yellow iron oxide-coated synthetic phlogopite ※2 | 20 |
| (component (A)) | |
| 10. Black iron oxide | 8 |

(Production method)
A. Components (1) to (3) and (7) to (10) were uniformly mixed.
B. Components (4) to (6) were uniformly mixed.

C. B was added to A to obtain a cosmetic base, 50 parts by mass of a water/ethanol mixed solution was added to 100 parts by mass of the cosmetic base, mixing and kneading were performed, and 2.5 g of the mixture was filled in a metal container (2.5 cm×2 cm×0.3 cm).

D. Two sheets of cellulose paper having a thickness of 0.02 mm were stacked on the surface of C, the water/ethanol mixed solution was removed by suction for 4 seconds while compression molding was performed at 2 kgf/cm² using a press mold from above, and then drying was performed at 70° C. for 8 hours to obtain a solid powder cosmetic (foundation).

The solid powder cosmetic (foundation) obtained as described above was satisfactory in all respects of absence of an appearance color difference, moldability, easiness of scooping, and matte texture.

The present application is based on Japanese Patent Application No. 2022-212766 filed on Dec. 28, 2022, the disclosure content of which is incorporated herein by reference in its entirety.

1. A solid powder cosmetic being obtained by adding a solvent to a cosmetic base containing the following components (A) and (B);
(A) a non-brilliant powder in which (a-2) a plate-like powder is coated with (a-1) a chromatic iron oxide, and
(B) a non-volatile oil,
to form a slurry, filling a container with the slurry, and then removing the solvent, wherein a chromatic iron oxide not coating a powder component is not contained or a content of the iron oxide is 15 mass % or less with respect to a total amount of the cosmetic,
a brilliant powder is not contained or a content of the brilliant powder is 15 mass % or less with respect to the total amount of the cosmetic, and
(a-1):(the chromatic iron oxide not coating the powder component) that is a mass content ratio of the (a-1) and the chromatic iron oxide not coating the powder component is 100:0 to 20:80.

2. The solid powder cosmetic according to claim 1, wherein the (a-2) is one or two or more kinds selected from mica and synthetic phlogopite.

3. The solid powder cosmetic according to claim 1, wherein the brilliant powder is not contained or the content of the brilliant powder with respect to the total amount of the cosmetic is 5 mass % or less.

4. The solid powder cosmetic according to claim 1, wherein the component (B) is one or two or more kinds selected from the group consisting of a hydrocarbon oil, a silicone oil, and an ester oil.

5. The solid powder cosmetic according to claim 4, wherein the ester oil contains malic acid diester and/or cetyl 2-ethylhexanoate.

6. The solid powder cosmetic according to claim 1, wherein the component (A) is a combination of red iron oxide-coated synthetic phlogopite and yellow iron oxide-coated synthetic phlogopite.

7. A method for producing a solid powder cosmetic obtained by adding a solvent to a cosmetic base containing the following components (A) and (B);
(A) a non-brilliant powder in which (a-2) a plate-like powder is coated with (a-1) a chromatic iron oxide, and
(B) a non-volatile oil,
to form a slurry, filling a container with the slurry, and then removing the solvent,
wherein a chromatic iron oxide not coating a powder component is not contained or a content of the iron oxide is 15 mass % or less with respect to a total amount of the cosmetic,
a brilliant powder is not contained or a content of the brilliant powder is 15 mass % or less with respect to the total amount of the cosmetic, and
(a-1):(the chromatic iron oxide not coating the powder component) that is a mass content ratio of the (a-1) and the chromatic iron oxide not coating the powder component is 100:0 to 20:80.
